(19) United States
(12) Patent Application Publication
(10) Pub. No.: US 2018/0296183 A1
(43) Pub. Date: Oct. 18, 2018

(54) METHOD AND APPARATUS FOR ULTRASOUND IMAGING OF BRAIN ACTIVITY

(71) Applicants: VIB VZW, Gent (BE); IMEC, Heverlee (BE); Katholieke Universiteit Leuven, K.U.Leuven R&D, Leuven (BE)

(72) Inventors: Alan Urban, Brussels (BE); Gabriel Montaldo, Brussels (BE); Jean Rossier, Paris (FR)

(73) Assignees: VIB VZW, Gent (BE); IMEC, Heverlee (BE); Katholieke Universiteit Leuven, K.U.Leuven R&D, Leuven (BE)

(21) Appl. No.: 15/524,251

(22) PCT Filed: Oct. 21, 2015
(86) PCT No.: PCT/EP2015/074343

§ 371 Date: May 3, 2017

(30) Foreign Application Priority Data
Nov. 4, 2014 (EP) 14306768.4

(51) Int. Cl. A61B 8/08; A61B 8/06; G01S 15/89

(52) U.S. Cl. CPC A61B 8/0816; A61B 8/06; A61B 8/5223; G01S 15/8915; G01S 15/8977; G01S 15/8995; A61B 8/5276

(57) ABSTRACT

Disclosed is a method for imaging brain activity from a set of ultrasound images I(t) of blood in a brain, wherein a measured spectrum s(P,t,ω) is computed at each point P of the ultrasound images, a reference spectrum s̅(P,ω) is determined at each point P, based on measured spectrums at point P, the reference spectrogram having a high frequency edge decaying in a frequency ωₘᵢₙ(P) to ωₘₐₓ(P), and a differential intensity is computed as:

dI(P,t)=∫_{ω}_{ₘᵢₙ}_{(P)}^{ω}^{ₘₐₓ}^{(P)}A(P,ω)[s(P,t,ω)−s̅(P,ω)]dω

wherein A(P,ω) is a positive weighting function.

### CROSS-REFERENCE TO RELATED APPLICATIONS

This application is a national phase entry under 35 U.S.C. § 371 of International Patent Application PCT/EP2015/074343, filed Oct. 21, 2015, designating the United States of America and published in English as International Patent Publication WO 2016/071108 A1 on May 12, 2016, which claims the benefit under Article 8 of the Patent Cooperation Treaty to European Patent Application Serial No. 14306768.4, filed Nov. 4, 2014.

### TECHNICAL FIELD

This application relates to methods and apparatuses for ultrasound imaging of brain activity.

### BACKGROUND

Brain activity can be imaged through imaging of hemodynamics, based on the phenomenon known as neurovascular coupling, which locally increases blood flow in an activated region of the brain.

Such imaging can be obtained by ultrasounds. Such ultrasound imaging has proved to be very efficient in terms of resolution, speed in obtaining the images (real-time imaging is possible), simplicity and cost (the imaging device is small and of relatively low cost compared to other methods such as magnetic resonance imaging (“MRI”)). Ultrasound imaging of brain hemodynamics and brain activity, i.e., functional imaging, has been described in particular by Macé et al:
“Functional ultrasound imaging of the brain: theory and basic principles,” IEEE Trans. Ultrason. Ferroelectr. Freq. Control, 2013 March; 60(3):492-506, and
“Functional ultrasound imaging of the brain,” Nature Methods, 8:662-664, 2011.

Such ultrasound functional imaging is usually based on ultrasound synthetic imaging as explained in the above publications and in EP2101191, wherein each ultrasound image is computed by compounding several ultrasound raw images that are obtained, respectively, by several emissions of plane ultrasonic waves in different directions.

The usual methods to detect the blood flow with ultrasound are the two classical Doppler modes: the color Doppler and the power Doppler. However, these methods lack sensitivity to efficiently detect neurovascular coupling.

### BRIEF SUMMARY

This disclosure, in particular, discloses improved existing imaging methods with improved sensitivity.

To this end, an embodiment of the disclosure relates to a method for imaging brain activity, including the following steps:
(a) an ultrasound imaging step wherein a set of ultrasound images I(t) of blood in a brain of a living subject are obtained at successive times t by transmission and reception of ultrasonic waves,
(b) a spectrum computing step wherein a measured spectrum s(P,t,ω) is computed at each point P of at least a region of at least some of the ultrasound images I(t), where ω is the frequency,
(c) a reference spectrum-determining step wherein a reference spectrum s̅(P,ω) is determined at each point P, based on at least one measured spectrum at point P, the reference spectrum having a high frequency edge decaying in at least a frequency band ωₘᵢₙ(P) to ωₘₐₓ(P),
(d) a differential intensity computing step wherein a differential intensity is computed as:

dI(P,t)=∫_{ω}_{ₘᵢₙ}_{(P)}^{ω}^{ₘₐₓ}^{(P)}A(P,ω)[s(P,t,ω)−s̅(P,ω)]^{r}dω

where r is a positive, non-zero number and A(P,ω) is a positive weighting function,
(e) a brain activity imaging step wherein an image of brain activity C(P) is determined based on the differential intensity.

The above differential intensity exhibits a very good signal-to-noise ratio and excellent sensitivity, enabling quick detection and reliable activation of functional zones in the brain, including under very low stimulus.

In addition, in various embodiments of the method of this disclosure, one may use one or more of the following arrangements:
at the reference spectrum determining step (c), the reference spectrum s̅(P,ω) is determined by averaging several measured spectra s(P,t,ω);
at the reference spectrum determining step (c), the reference spectrum s̅(P,ω) is determined by approximating an average sₘ(P,t,ω) of at least one measured spectrum s(P,t,ω) by a substantially square function having a flat central portion, a low frequency edge and a high frequency edge;
the flat central portion of the substantially square function is between two frequencies ω₁ and ω₂, which are such that sₘ(P, ω) is more than a predetermined value x between ω₁ and ω₂, x being a positive number greater than 0.3 and lower than 0.8, and ω₁<ω₂;
the high frequency edge is decaying such that:

s̅(P,ω)=λSu(ω·ω₀/ω₂) for ω>ω₂

where:
Su is the spectrum of the ultrasonic waves,
ω₀ is a central frequency of the ultrasonic waves, and
λ is a positive, non-zero scale factor.
the low frequency edge is decaying such that:

s̅(P,ω)=λ′H(ω) for ω<ω₁

where:
H(ω) is a transfer response of a filter applied to the ultrasound images to eliminate the movements of tissues,
λ is a positive, non-zero scale factor;
the weighting function A(P,ω) is determined as:

$$A ( P , ω ) = \frac{\partial \overline{s} ( P , ω ) / \partial ω}{{σ}^{2} ( P )} ,$$

where σ(P) is the standard deviation of s̅(P,ω) at point P;
the weighting function A(P,ω) is a square function;
ωₘᵢₙ(P) is such that s(P,ωₘᵢₙ(P))/s̅ₘₐₓ(P) is in the range 0.8 to 1, ωₘₐₓ(P) is such that s(P,ωₘₐₓ(P))/s̅ₘₐₓ(P) is in the range 0 to 0.5, and s̅ₘₐₓ(P) is a maximum of s̅(P,ω);
ωₘᵢₙ(P) is such that s(P,ωₘᵢₙ(P))/s̅ₘₐₓ(P) is in the range 0.8 to 0.99, ωₘₐₓ(P) is such that s(P,ωₘₐₓ(P))/s̅ₘₐₓ(P) is in the range 0.01 to 0.3;
ωₘᵢₙ(P) is such that s(P,ωₘᵢₙ(P))/s̅ₘₐₓ(P) is in the range 0.85 to 0.95, ωₘₐₓ(P) is such that s(P,ωₘₐₓ(P))/s̅ₘₐₓ(P) is in the range 0.01 to 0.1;
the ultrasound imaging step (a) includes:
   (a2) A raw imaging step in which raw images Iᵣ(t) of the living tissues (1) are taken at successive times t by transmission and reception of ultrasonic waves,
   (a2) a filtration step in which each raw image Iᵣ(t) is filtered to eliminate the movements of tissues and obtain the ultrasound image I(t);
the image C(P) of brain activity computed at step (d) is obtained by correlation with a predefined temporal stimulation signal stim(t) applied to the subject;
the image C(P) of brain activity is computed as:

$$C ( P ) = \int dInorm ( P , t ) stim ( t ) dt$$

$$wherein : \\ dInorm ( x , z , t ) = \frac{dI ( P , t ) - dI 0 ( P )}{\sqrt{\int {( dI ( P , t ) - dI 0 ( P ) )}^{2} dt}} , \\ dI 0 ( P ) = \int dI ( P , t ) dt ;$$

$$r = 1.$$

In addition, another embodiment of the disclosure relates to an apparatus for imaging brain activity, adapted to:
(a) take a set of ultrasound images I(t) of blood in a brain of a living subject at successive times t by transmission and reception of ultrasonic waves,
(b) computing a measured spectrum s(P,t,ω) at each point P of at least a region of at least some of the ultrasound images I(t), where ω is the frequency,
(c) determine a reference spectrum s̅(P,ω), which is determined at each point P, based on at least one measured spectrum at each point P, the reference spectrum having a high frequency edge decaying in at least a frequency band ωₘᵢₙ(P) to ωₘₐₓ(P),
(d) computing a differential intensity as:

dI(P,t)=∫_{ω}_{ₘᵢₙ}_{(P)}^{ω}^{ₘₐₓ}^{(P)}A(P,ω)[s(P,t,ω)−s̅(P,ω)]^{r}dω

where r is a positive, non-zero number and A(P,ω) is a positive weighting function,
(e) determine an image of brain activity C(P) based on the differential intensity.

### BRIEF DESCRIPTION OF THE DRAWINGS

Other features and advantages of the embodiments of the disclosure appear from the following detailed description of one embodiment thereof, given by way of non-limiting example, and with reference to the accompanying drawings.

In the drawings:

FIG. 1 is a schematic drawing showing an ultrasound imaging device according to one embodiment of the disclosure;

FIG. 2 is a block diagram showing part of the apparatus of FIG. 1;

FIG. 3, Panel a, shows image of blood intensity of a rat brain and a detail thereof showing one particular selected micro-vessel, which can be obtained by the apparatus of FIGS. 1 and 2;

FIG. 3, Panel b, is a spectrogram of the selected micro-vessel of FIG. 3, Panel a;

FIG. 3, Panel c, c is a spectrum of the selected micro-vessel, compared to a theoretical model thereof;

FIG. 4, Panel a, is a spectrogram of the selected micro-vessel showing the evolution of the signal frequency during a nervous stimulation;

FIG. 4, Panel b, is spectrum corresponding to the frequency signal of FIG. 4, Panel a, before and during stimulation;

FIG. 4, Panel c, shows the noise as a function of the frequency in the spectrum of FIG. 4, Panel b;

FIG. 4, Panel d, shows the intensity as a function of the frequency in the spectrum of FIG. 4, Panel c;

FIG. 5, Panels a and b, show, respectively, a map of intensity and a map of differential intensity computed according to the disclosure, based on the same ultrasonic image taken after stimulation by a short pulse.

### DETAILED DESCRIPTION

In the figures, the same references denote identical or similar elements.

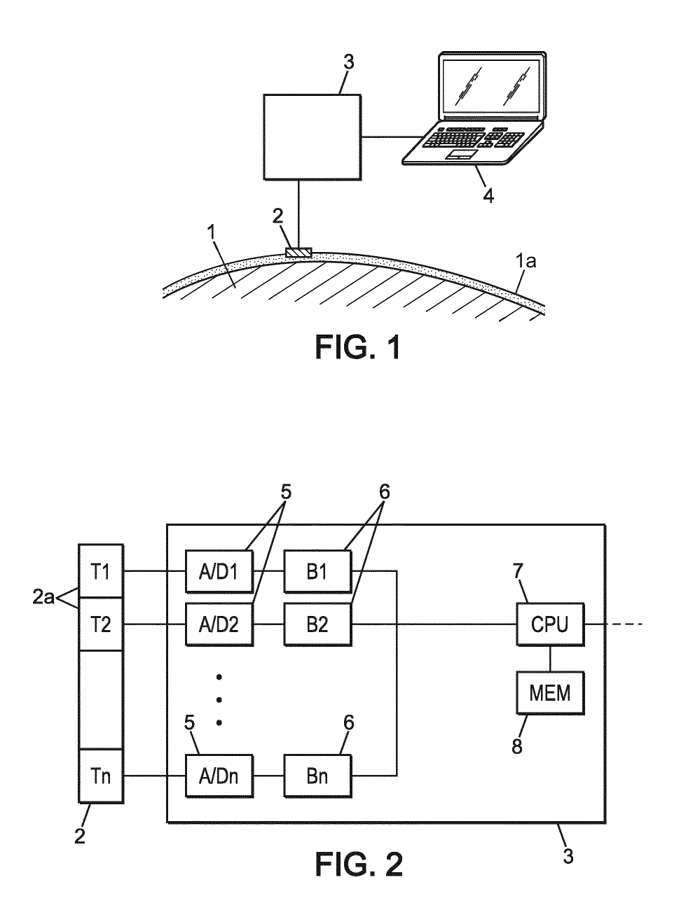

The apparatus shown in FIG. 1 is adapted for ultrasound imaging of living tissues 1, in particular, human or animal tissues. The living tissues 1 may be, in particular, a brain or part of a brain.

The apparatus may include, for instance, as illustrated in FIGS. 1 and 2:
an ultrasound transducer array 2 (T₁-Tₙ), for instance, a linear array typically including a few tens of transducers (for instance, 100 to 300) juxtaposed along an axis as already known in usual echographic probes (the array 2 is then adapted to perform a bidimensional (2D) imaging of the tissues 1, but the array 2 could also be a bidimensional array adapted to perform a tridimensional (3D) imaging of the tissues 1); the transducers in the array 2 may, for instance, transmit and receive ultrasound waves of frequencies usually between 2 and 40 MHz; in the case of the brain, transmission and reception can be performed through the skull 1a or directly in contact with the brain tissues 1, e.g., at one or several aperture(s) provided in the skull;
an electronic control circuit 3 controlling the transducer array 2 and acquiring signals therefrom;
a computer 4 or similar for controlling the electronic circuit 3 and viewing ultrasound images obtained from the control circuit 3 (in a variant, a single electronic device could fulfill all the functionalities of the electronic control circuit 3 and of the computer 4).

As shown in FIG. 2, the electronic control circuit 3 may include, for instance:
n analog/digital converters 5 (A/D1-A/Dn) individually connected to the n transducers (T1-Tn) of the transducer array 2;
n buffer memories 6 (B1-Bn) respectively connected to the n analog/digital converters 5;
a central processing unit 7 (CPU) communicating with the buffer memories 6 and the computer 4;
a memory 8 (MEM) connected to the central processing unit 7.

A new method for imaging brain activity, which may use the above apparatus, will now be described. This method may include an ultrasound imaging step (a), a spectrogram computing step (b), a reference spectrogram-determining step (c), a differential intensity computing step (d), and a brain activity imaging step (e).

### (a) Ultrasound Imaging Step:

(a1) Raw Imaging Step:

A step in which raw images Iᵣ(t) of the living tissues (1) are taken at successive times t by transmission and reception of ultrasonic waves.

The apparatus of FIGS. 1 and 2 may be adapted to perform synthetic ultrasound imaging as described by Macé et al. (“Functional ultrasound imaging of the brain: theory and basic principles,” IEEE Trans. Ultrason. Ferroelectr. Freq. Control, 2013 March; 60(3):492-506, and “Functional ultrasound imaging of the brain,” Nature Methods, 8:662-664, 2011), and EP2101191. In that case, each ultrasound image is computed by compounding several ultrasound raw images that are obtained, respectively, by several emissions of plane ultrasonic waves in different directions. For instance, the several ultrasound raw images can be acquired at a rate of 2500 raw images per second, with cyclic variations of, e.g., 5° in the direction of propagation of the plane waves (−10°, −5°, 0°, +5°, +10°), wherein five raw ultrasound images are compounded to do one ultrasound (compounded) image. In that case, the ultrasound (compounded) images are produced at a rate of 500 images per second.

In any case, a set of N ultrasound images I(tₖ) of the living tissues is taken at successive times tₖ (here, for instance, every 2 ms), by the above method of synthetic imaging or otherwise. N can usually comprise between 200 and 30,000; for instance, N may be between 1500 and 2500, e.g., around 2000.

When the array 2 is linear, each image I(tₖ) is a bidimensional matrix I(tₖ)=(I₁ₘ(tₖ)), where the component I₁ₘ(tₖ) of this matrix is the value of the pixel l,m of abscise x₁ along the array 2 and of ordinate zₘ in the direction of the depth. For instance, the pixels may be 90 spaced every 50 μm in depth and 128 spaced every 100 μm in abscise.

In the following, the images I will be indifferently presented either in the above matrical notation I(tₖ)=(I₁ₘ(tₖ)), or in continuous notation I(x,z,t).

(a2) Filtration Step:

The following filtration step is optional only in this disclosure; it may be avoided or replaced by another filtration.

The images I(tₖ) are the sum of a tissular component Iₜᵢₛₛ(tₖ) and a vascular component I_{blood}(tₖ) due to the blood flow:

I(tₖ)=Iₜᵢₛₛ(tₖ)+I_{blood}(tₖ) (1)

To compute a hemodynamic image of the tissues, it is necessary to eliminate the tissular component Iₜᵢₛₛ(tₖ), since the tissues have slow movements of similar velocity to the blood flow in the smallest vessels (capillary and arterioles).

This filtration process may be carried out, for instance, in three sub-steps (a21) to (a23) as explained below. However, any of these sub-steps could be omitted or replaced by a different filtration.

(a21) Elimination of the Fixed Tissues:

In a first substep (a21), a same fixed image I1 (for instance, I1=I(t=0)) can be subtracted from all images I(tₖ). For more simplicity, the image after subtraction of I1 will still be named I(tₖ) hereafter.

(a22) High Pass Filter:

In a second substep (a22), a highpass temporal filter may be applied to the images I(tₖ). This highpass temporal filter may have a cut-off frequency less than 15 Hz, for instance, the cut-off frequency may be 5 to 10 Hz.

More generally, the cut-off frequency will be less than 5·10⁻⁶·f_{US}, where f_{US} is the frequency of the ultrasonic waves.

For more simplicity, the image after application of the high pass filter will still be named I(tₖ) hereafter.

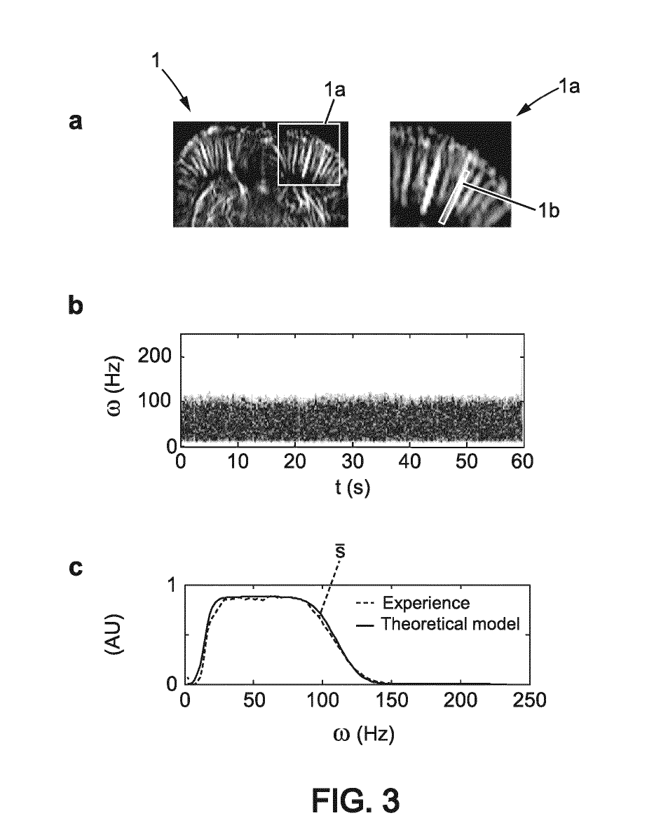

The high pass filter eliminates part of the tissular component Iₜᵢₛₛ(tₖ) of the images I(tₖ), corresponding to axial velocities (perpendicular to the array 2) less than 0.5 mm/s in the case of a cutoff frequency of 10 Hz, as shown in FIG. 3, Panel b. This high pass filter leaves substantially intact the vascular component I_{blood}(tₖ), specially compared to the high pass filter of the prior art with a cutoff frequency of 75 Hz, which eliminated all blood flows having a velocity less than 3.75 mm per s.

(a23) Spatiotemporal Filter:

Complete elimination of the tissular component Iₜᵢₛₛ(tₖ) is done by a spatiotemporal filter applied to the image I(tₖ), after substeps (a21) and/or (a22) or directly after step (a). This spatiotemporal filter is based on a physical difference between a vascular signal and a tissue movement: the tissue movement is coherent at least at small scale, whereas the blood flows are not.

As a matter of fact, a movement is propagated in the tissue by mechanical waves whose speeds are ˜1 m/s for the shear waves and 1500 m/s for the compression waves (in the case of the brain). The wavelength of these mechanical waves is very high compared to the size of the blood vessels; for example, a wave of 100 Hz has a wavelength of 1 cm for the shear wave and 15 m for the compression wave. Accordingly, all the tissue at the scale of 1 cm moves coherently.

On the contrary, the vascular signal comes from the movement of red blood cells that flow randomly inside the vessel and generate a signal that is uncorrelated between two different pixels.

Based in this difference, the tissular component Iₜᵢₛₛ(tₖ) can be filtered by determining a spatially correlated component Iₜᵢₛₛ(tₖ) corresponding to spatially coherent movements of the tissues, and the spatially correlated component Iₜᵢₛₛ(tₖ) is subtracted from the image I(tₖ) so as to determine a filtered image I_{f}(tₖ)=I(tₖ)−Iₜᵢₛₛ(tₖ).

To summarize, Iₜᵢₛₛ(tₖ) may be determined such that:

Iₜᵢₛₛ(tₖ)=a(tₖ)I₀ (2),

wherein a(tₖ) is a real number function of time and I₀ is a fixed image of the tissues.

For a given point P (pixel) in the image I(tₖ), the spatially coherent component Iₜᵢₛₛ(tₖ) may be determined in an adjacent area A(P) around the given point P, the area A(P) not covering the whole image I(tₖ). For instance, the adjacent area A(P) may have between 10 and 200 pixels, for instance, 10*10 pixels.

The spatially coherent component Iₜᵢₛₛ(tₖ) may be determined by various mathematical methods, for instance, by recurring estimates, or by the following method.

A practical method to determine the spatially coherent component Iₜᵢₛₛ(tₖ) is to decompose the images, image I(tₖ) using a singular value decomposition (SVD). FIG. 3, Panel a, shows the distribution of the singular values in a particular example of ultrasound imaging performed on the brain of a living rat. This distribution is mainly continuous, with 12 exceptional high values that are outside the main continuous distribution. By eliminating these outside values or at least the highest one or the N_{f} highest ones (N_{f} being a non-zero positive integer), the spatially coherent component Iₜᵢₛₛ(tₖ) can be eliminated.

More precisely, for each given point P in the image I(t), the coherent component I_{tiss,A}(tₖ) in the adjacent area A(P) around the given point P, is determined in the form:

I_{tiss,A}(tₖ)=Σᵢ₌₁^{Nf}λᵢmᵢsᵢ(tₖ) (3),

wherein:
λᵢ are the N_{f} highest singular value(s) of the images I(tₖ) in the adjacent area A(P), ordered, e.g., by decreasing order,
mᵢ are constant images covering the area A(P) and Sᵢ(tₖ) is a complex number function of time, m₁ S₁(tₖ) to m_{Nf} S_{Nf}(tₖ) corresponding to the N_{f} highest singular value(s) of the images I(tₖ) in the adjacent area A(P).

In practice, elimination of the highest singular values can often be limited to N_{f}=2 or 3, or even to 1, in which case:

I_{tiss,A}(tₖ)=λ₁m₁s₁(tₖ) (3′),

A value in time of Iₜᵢₛₛ(tₖ) at point P is then determined as the value of I_{tiss,A}(tₖ) at point P. The filtered image signal of blood at point P is then determined based on equation (1):

I_{blood}(tₖ)=I(tₖ)−I_{tiss,A}(tₖ) (1′).

To perform the SVD, all the images I(tₖ) may be gathered into a single bidimensional matrix M=M(p,k), wherein M_{p,k}=I₁ₘ(tₖ)), l,m being two indexes representing the position in the image I(tₖ), p being an index bijectively connected to each pair of indexes l,m; p can be computed in the form:

p=l−m·nₓ (4),

where nₓ is the number of pixels in a line parallel to the array 2 of transducers.

Thus, the SVD is done on matrix M and N_{f} highest singular values are eliminated from M to obtain a filtrated matrix M_{f}. The filtrated images I_{f}(tₖ) are then determined from Mf, based on the above formula (4), which enables finding indexes l and m based on index p.

FIG. 3, Panel a, shows one example of Doppler image of the brain 1 of a living rat, obtainable from the ultrasound image of step (a). A detail of a region of interest 1a belonging to the cortical part, is also shown in FIG. 3, Panel a, where a selected vessel 1b can be seen.

### (b) Spectrum Computing Step

Starting from the set of ultrasound images I(t) of blood obtained at the imaging step (a), a measured spectrogram spg(P,t) can be computed for at least some points P. FIG. 3, Panel b, shows an example of a measured spectrogram spg(P,t) for a particular point P in the vessel 1b of FIG. 3, Panel a.

A measured spectrum s(P,t,ω) (where ω is the frequency) is computed at each point P of at least a region of at least some of the ultrasound images I(t). This spectrum can be, for instance, a sliding or window spectrum that is computed in each pixel P(x,z) as:

$$s ( P , t , ω ) = \int I ( P , {t}^{′} ) W ( \frac{t - {t}^{′}}{2 T} ) {e}^{i ω {t}^{′}} {dt}^{′} \qquad ( 5 )$$

where W is a square window function and T is the length of the window.

### (c) Reference Spectrum-Determining Step

A reference spectrum s̅(P,ω) is determined at each point P, based on at least one measured spectrum at point P, the reference spectrum having a high frequency edge decaying in at least a frequency band ωₘᵢₙ(P) to ωₘₐₓ(P).

The reference spectrum s̅(P,ω) can be determined, for instance, by averaging several measured spectra s(P,t,ω), for instance, at least 10 measured spectra, usually 10 to 20 measured spectra:

$$\overline{s} ( P , ω ) = {s}_{m} ( P , ω ) = \frac{1}{n} {\sum }_{i = 1}^{n} s ( P , {t}_{i} , ω ) , \qquad ( 6 )$$

where n is the number of measured spectra in the average.

More generally, such mean spectrum may be expressed as:

$$\overline{s} ( P , ω ) = {s}_{m} ( P , ω ) = \frac{1}{{T}_{tot}} \int [ s ( P , t , ω ) ] d ω \qquad ( 6 a )$$

where Tₜₒₜ is the duration of integration of s(P,t,ω).

In a particular case, the mean spectrum s̅(P,ω) can be simply one of the measured spectra s(P,t0,ω) (t0 being one of the instants of measurement) in the absence of excitation applied to the considered functional zone of the brain. Thus, in the most general case, the mean spectrum is obtained by averaging a group of at least one measured spectra.

FIG. 3, Panel c, shows in dotted lines a reference spectrum s̅(P,ω) computed at the above-mentioned point P in the vessel 1b, by the above averaging method. FIG. 3, Panel c, also shows in solid line, an example of spectrum computed from a theoretical model as taught by Censor et al. (IEEE TRANSACTIONS ON Biomedical Engineering, Vol. 35, No. 9, September 1988), which is remarkably in line with the experimental reference spectrum in dotted lines.

In a particularly advantageous variant, the reference spectrum s̅(P,ω) can be obtained by approximating such mean spectrum sₘ(P,ω) as defined above, by a substantially square function having a flat central portion and two edges, which can be either sharp, or preferably decaying. For instance, the flat central portion of s̅(P,ω) can be equal to 1. Advantageously, the flat central portion of s̅(P,ω) is between two frequencies ω₁ and ω₂, which are such that sₘ(P, ω) is more than a predetermined value x between ω₁ and ω₂, x being a positive number greater than 0.3 and lower than 0.8 (for instance x=0.5) and sₘ(P, ω₁)=sₘ(P,ω₂)=x, and ω₁<ω₂.

In the case of decaying edges, one edge could be sharp and the other edge decaying; for instance, the high frequency edge (for ω>ω₂) could be the only decaying edge.

In the case of decaying edges, one possibility for the high frequency decaying edge (for ω>107 ₂), is to have the same shape than the spectrum of the emitted ultrasound signal, with a scale factor. If Su(ω) is the spectrum of the emitted ultrasound signal, the high frequency edge can be of the shape:

s̅(P,ω)=λSu(ω·ω₀/ω₂) for ω>ω₂ (7)

where ω₀ is the central frequency of the ultrasounds and λ is a positive, non-zero scale factor, chosen, for instance, such that s̅(P,ω₂)=λA(ω₀)=x.

Again, in the case of decaying edges, one possibility for the low frequency decaying edge is to use the transfer response H(ω) of the filter used to eliminate the signal from the tissues and thus select the blood signal, at step (a2). Thus, the low frequency decaying edge can be in the form:

s̅(P,ω)=λ′H(ω) for ω<ω₁ (8)

where λ′ is a positive, non-zero scale factor, chosen, for instance, such that s̅(P,ω₁)=λ′H(ω₁)=x.

### (d) Differential Intensity Computing Step

A differential intensity dI(P,t) can then be computed for at least some instances t, as:

dI(P,t)=∫_{ω}_{ₘᵢₙ}_{(P)}^{ω}^{ₘₐₓ}^{(P)}A(P,ω)[s(P,t,ω)−s̅(P,ω)]^{r}dω (9)

where r is a positive, non-zero number and A(P,ω) is a positive weighting function.

Advantageously, r=1 (this case will be considered hereafter in the description). This power r could also be 2, for instance.

The weighting function A(P,ω) can be determined, for instance, as:

$$A ( P , ω ) = \frac{\partial \overline{s} ( P , ω ) / \partial ω}{{σ}^{2} ( P , ω )} \qquad ( {9}^{′} )$$

where σ(P) is the standard deviation of s̅(P,ω) at point P:

σ²(Pω)=∫(s(P,ω,t)−s̅(P,ω,t)²)dt (10)

The weighting function A(P,ω) can be a square function.

ωₘᵢₙ(P) and ωₘₐₓ(P) can be determined, for instance, as follows:
ωₘᵢₙ(P) is such that s(P,ωₘᵢₙ(P))/s̅ₘₐₓ(P) is in the range 0.8 to 1,
ωₘₐₓ(P) is such that s(P,ωₘₐₓ(P))/s̅ₘₐₓ(P) is in the range 0 to 0.5,
s̅ₘₐₓ(P) is a maximum of s̅(P,ω).

In a particular embodiment, ωₘᵢₙ(P) and ωₘₐₓ(P) can be determined as follows:
ωₘᵢₙ(P) is such that s(P,ωₘᵢₙ(P))/s̅ₘₐₓ(P) is in the range 0.8 to 0.99,
ωₘₐₓ(P) is such that s(P,ωₘₐₓ(P))/s̅ₘₐₓ(P) is in the range 0.01 to 0.3.

In a more particular embodiment, ωₘᵢₙ(P) and ωₘₐₓ(P) can be determined as follows:
ωₘᵢₙ(P) is such that s(P,ωₘᵢₙ(P))/s̅ₘₐₓ(P) is in the range 0.85 to 0.95,
ωₘₐₓ(P) is such that s(P,ωₘₐₓ(P))/s̅ₘₐₓ(P) is in the range 0.01 to 0.1.

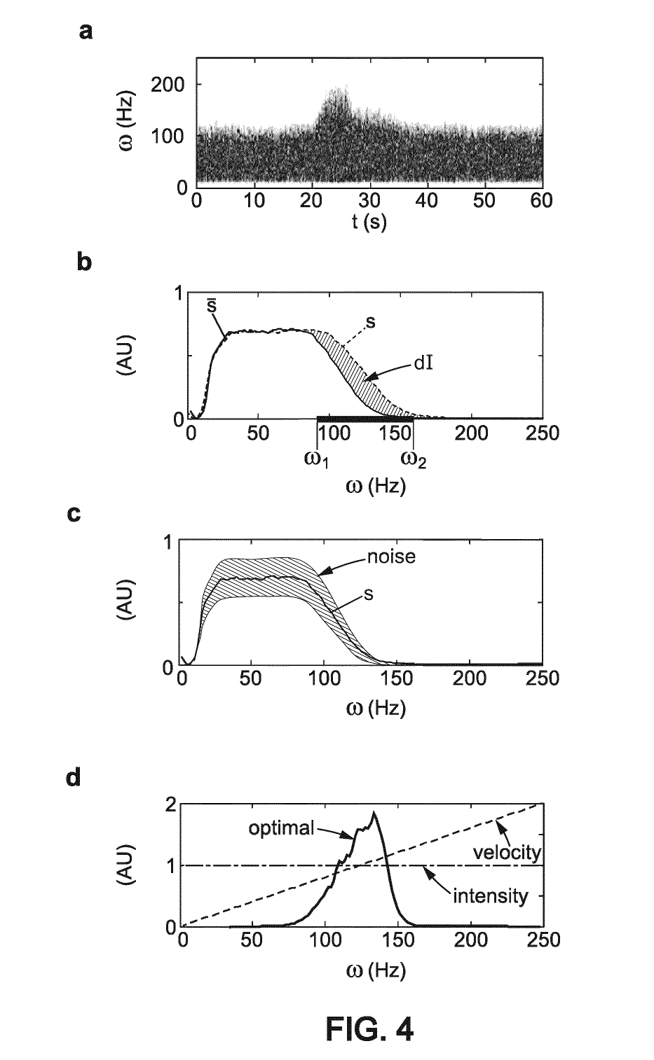

When the brain is activated, the velocity of blood increases and modifies the spectrogram as shown in FIG. 4, Panel a. During the activation, the maximal frequency increases and the spectrum s is dilated as shown in FIG. 4, Panel b. The area between the activated spectrum s(P,ω) and the reference spectrum s̅(P,ω) is the above differential intensity dI.

As shown in FIG. 4, Panel c, one of the advantages of the differential intensity dI is that it is computed on the part of the spectrum, which exhibits the least noise, so the best signal-to-noise ratio.

FIG. 4, Panel d, shows the optimal weighting function A(P,ω) as explained above

$$( A ( P , ω ) = \frac{\partial \overline{s} ( P , ω ) / \partial ω}{{σ}^{2} ( P , ω )} ) ,$$

compared to the cases where velocity of the blood (usually Doppler, also called color Doppler, corresponding to A(P,ω)=ω) or intensity (power Doppler, corresponding to A(P,ω)=1) are used.

### (e) Brain Activity Imaging Step

An image of brain activity C(P) is then determined based on the differential intensity.

The image C(P) of brain activity can be obtained by correlation with a predefined temporal stimulation signal stim(t) applied to the subject. In particular, the image C(P) of brain activity can be computed as:

$$C ( P ) = \int dInorm ( P , t ) stim ( t ) dt$$

$$wherein : \\ dInorm ( x , z , t ) = \frac{dI ( P , t ) - dI 0 ( P )}{\sqrt{\int {( dI ( P , t ) - dI 0 ( P ) )}^{2} dt}} ,$$

and dI0(P)=˜dI(P,t)dt is the continuous component.

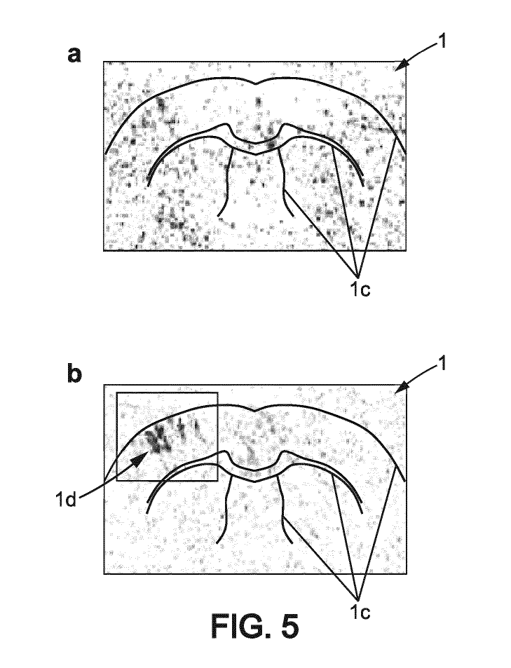

FIG. 5, Panel b, shows an example of such brain activation image for a very small electrical stimuli in the forepaw of only 200 μs. The image clearly shows activated zones 1d.

FIG. 5, Panel a, shows an activation image computed with intensity according to the prior art, from the same stimulus and the same measurement: no activated zone can be seen.

1. A method for imaging brain activity, the method comprising:
obtaining a set of ultrasound images I(t) of blood in a brain of a living subject at successive times t by transmission and reception of ultrasonic waves;
computing a measured spectrum s(P,t,ω) at each point P of at least a region of at least some of the ultrasound images I(t), wherein ω is the frequency;
determining a reference spectrogram s̅(P,ω) at each point P, based on at least one measured spectrum at point P, said reference spectrum having a high frequency edge decaying in at least a frequency band; and
computing a differential intensity,
dI(P,t)=∫_{ω}_{ₘᵢₙ}_{(P)}^{ω}^{ₘₐₓ}^{(P)}A(P,ω)[s(P,t,ω)−s̅(P,ω)]^{r}dω
wherein r is a positive, non-zero number and A(P,ω) is a positive weighting function,
determining an image of brain activity C(P) based on said differential intensity.

2. The method of claim 1, further comprising determining said reference spectrum s̅(P,ω) by averaging a plurality of measured spectra s(P,t,ω).

3. The method of claim 1, further comprising determining said reference spectrum s̅(P,ω) by approximating an average sₘ(P,t,ω) of at least one measured spectrum s(P,t,ω) by a substantially square function having a flat central portion, a low frequency edge and a high frequency edge.

4. The method of claim 3, wherein the flat central portion of said substantially square function is between two frequencies ω₁ and ω₂ which are such that sₘ(P, ω) is more than a predetermined value x between ω₁ and ω₂, x being a positive number greater than 0.3 and lower than 0.8, and ω₁<ω₂.

5. The method of claim 4, wherein said high frequency edge is decaying such that:
s̅(P,ω)=λSu(ω,ω₀/ω₂) for ω>ω₂
wherein
Su is the spectrum of the ultrasonic waves,
ω₀ is a central frequency of the ultrasonic waves, and
λ is a positive, non-zero scale factor.

6. The method of claim 4, wherein said low frequency edge is decaying such that
s̅(P,ω)=λ′H(ω) for ω<ω₁
wherein:
H(ω) is a transfer response of a filter applied to the ultrasound images to eliminate the movements of tissues,
λ′ is a positive, non-zero scale factor.

7. The method of claim 1, wherein said weighting function A(P,ω) is determined as:
$$A ( P , ω ) = \frac{\partial \overline{s} ( P , ω ) / \partial ω}{{σ}^{2} ( P )} ,$$
wherein σ(P) is the standard deviation of s̅(P,ω) at point P.

8. The method of claim 1, wherein said weighting function A(P,ω) is a square function.

9. The method of claim 1, wherein:
ωₘᵢₙ(P) is such that s(P,ωₘᵢₙ(P))/s̅ₘₐₓ(P) is in the range 0.8 to 1,
ωₘₐₓ(P) is such that s(P,ωₘₐₓ(P))/s̅ₘₐₓ(P) is in the range 0 to 0.5,
s̅ₘₐₓ(P) is a maximum of s̅(P,ω).

10. The method of claim 9, wherein:
ωₘᵢₙ(P) is such that s(P,ωₘᵢₙ(P))/s̅ₘₐₓ(P) is in the range 0.8 to 0.99,
ωₘₐₓ(P) is such that s(P,ωₘₐₓ(P))/s̅ₘₐₓ(P) is in the range 0.01 to 0.3.

11. The method of claim 9, wherein:
ωₘᵢₙ(P) is such that s(P,ωₘᵢₙ(P))/s̅ₘₐₓ(P) is in the range 0.85 to 0.95,
ωₘₐₓ(P) is such that s(P,ωₘₐₓ(P))/s̅ₘₐₓ(P) is in the range 0.01 to 0.1.

12. The method of claim 1, wherein obtaining a set of ultrasound images comprises:
taking raw images Iᵣ(t) of said living tissues at successive times t by transmission and reception of ultrasonic waves,
filtering each raw image Iᵣ(t) to eliminate movements of tissues and obtain said ultrasound image I(t).

13. The method of claim 1, wherein obtaining the image C(P) of brain activity is obtained by correlation with a predefined temporal stimulation signal stim(t) applied to the subject.

14. The method of claim 13, wherein the image C(P) of brain activity is computed as:
$$C ( P ) = \int dInorm ( P , t ) stim ( t ) dt$$
$$wherein : \\ dInorm ( x , z , t ) = \frac{dI ( P , t ) - dI 0 ( P )}{\sqrt{\int {( dI ( P , t ) - dI 0 ( P ) )}^{2} dt}} , \\ dI 0 ( P ) = \int dI ( P , t ) dt - .$$

15. The method of claim 1, wherein r=1.

16. An imaging apparatus for imaging brain activity, comprising:
an ultrasonic transducer array that is configured to transmit and receive ultrasonic waves; an image processor coupled to the ultrasonic transducer array and configured to:
take a set of ultrasound images I(t) of blood in a brain of a living subject at successive times t responsive to the ultrasonic waves,
compute a measured spectrum s(P,t,ω) at each point P of at least a region of at least some of the ultrasound images I(t), where ω is the frequency,
determine a reference spectrum s̅(P,ω)is determined at each point P, based on at least one measured spectrum at each point P, said reference spectrum having a high frequency edge decaying in at least a frequency band ωₘᵢₙ(P) to ωₘₐₓ(P),
compute a differential intensity as:
dI(P,t)=∫_{ω}_{ₘᵢₙ}_{(P)}^{ω}^{ₘₐₓ}^{(P)}A(P,ω)[s(P,t,ω)−s̅(P,ω)]^{r}dω
wherein r is a positive, non-zero number and A(P,ω) is a positive weighting function, and
determine an image of brain activity C(P) based on said differential intensity.

17. The imaging apparatus of claim 16, wherein the image processor is further configured to determine said reference spectrum by averaging a plurality of measured spectra.

18. The imaging apparatus of claim 16, wherein the image processor is further configured to determine said reference spectrum by approximating an average of at least one measured spectrum by a substantially square function having a flat central portion, a low frequency edge and a high frequency edge.

19. The imaging apparatus of claim 16, wherein the flat central portion of said substantially square function is between two frequencies ω₁ and ω₂ which are such that sₘ(P, ω) is more than a predetermined value x between ω₁ and ω₂, x being a positive number greater than 0.3 and lower than 0.8, and ω₁<ω₂.

20. The imaging apparatus of claim 18, wherein said low frequency edge is decaying such that:
s̅(P,ω)=λ′H(ω) for ω<ω₁
wherein:
H(ω) is a transfer response of a filter applied to the ultrasound images to eliminate the movements of tissues,
λ′ is a positive, non-zero scale factor.
